(19) United States
(12) Patent Application Publication
(10) Pub. No.: US 2025/0302381 A1
(43) Pub. Date: Oct. 2, 2025

(54) USE OF A VARIABILITY METRIC OF GLUCOSE LEVEL VALUES TO IDENTIFY A SLEEPING TIME FRAME FOR A USER OF A MEDICAMENT DELIVERY DEVICE

(71) Applicant: INSULET CORPORATION, Acton, MA (US)

(72) Inventors: Mert SEVIL, Manchester, NH (US); Yibin ZHENG, Hartland, WI (US); William WHITELEY, Knoxville, TN (US)

(21) Appl. No.: 19/091,353

(22) Filed: Mar. 26, 2025

Related U.S. Application Data
(60) Provisional application No. 63/572,623, filed on Apr. 1, 2024.

(51) Int. Cl. A61B 5/00; G16H 50/20

(52) U.S. Cl. CPC A61B 5/4839; G16H 50/20

(57) ABSTRACT

A variability metric of glucose level values of a user is calculated for each candidate sleeping time frame in a data set of glucose level values of the user obtained over time for a specified period for each day of multiple days. Each of the candidate sleeping time frames may represent a duration in which the user may have been sleeping. Execution of the programming instructions further may cause the processor to determine a mean or median of the variability metric of the glucose level values in the data set for each candidate sleeping time frame across the days in the specified period, designate a selected one of the candidate sleeping time frames that has the lowest mean or median variability metric as the sleeping time frame, and configure the pump to deliver a sleeping basal rate of the medicament for the designated sleeping time frame.

### CROSS-REFERENCE TO RELATED APPLICATIONS

This application claims priority to and the benefit of U.S. Provisional Application No. 63/572,623, filed Apr. 1, 2024, the entirety of which is incorporated herein by reference.

### BACKGROUND

In a typical conventional automated insulin delivery (AID) system, a baseline basal delivery rate is input to a control system of the AID system and used by the control system in setting basal medicament delivery doses. This baseline basal rate may be based upon the total daily insulin (TDI) of the user. TDI represents the sum of insulin (both basal and bolus) that is delivered to the user in a day. The TDI may be chosen based on clinical parameters, such as gender, weight, and age. In many instances, the basal amount per day may be determined as a proportion of the TDI (e.g. 0.5, 0.45, or 0.6 of TDI). An hourly baseline basal rate may then be determined by dividing the basal amount per day by 24 (i.e., the number of hours in a day).

The control system may start the basal deliveries to the user at the baseline basal rate. The control system then may adjust the baseline basal rate to attempt to keep the glucose level of the user at a target glucose level. These adjustments may be made an ongoing basis, such as at each operational cycle of the AID system (e.g., every five minutes). The control system may predict what glucose levels will be an operational cycle in the future and choose a basal medicament dose for the next operational cycle based on the anticipated deviation of the one or more predicted glucose level and the target glucose level. The chosen basal medicament dose may constitute an adjustment to the dose derived the baseline basal delivery rate. This process is then repeated for each subsequent operational cycle. Hence, when a user is awake and when a user is sleeping, the control system may adjust the basal medicament delivery rate on an ongoing basis.

Determining an accurate basal medicament delivery rate for users is critical for AID systems. If the basal medicament delivery rate is too great for a user, there is a risk of hypoglycemia, whereas if the basal medicament delivery rate is too small for the user, there is a risk of hyperglycemia. Unfortunately, the basal proportion of total daily insulin for many users (i.e., the portion of TDI matches the user's true daily basal insulin needs) varies from person to person.

One challenge in establishing basal insulin rates is that basal insulin needs vary over the course of a day. The basal insulin needs of a user are greater during the day than at night. As a result, a fixed basal delivery rate, such as that derived from TDI, may be too high for the night when a user's basal insulin needs are lower and too low for the day when a user's basal insulin needs are higher.

### SUMMARY

In accordance with an inventive facet, a medicament delivery system may include a pump for delivering a medicament to a user and a storage for storing programming instructions. The medicament delivery system also may include a processor configured for executing the programming instructions. The execution of the programming instructions by the processor may cause the processor to calculate a variability metric of glucose level values of the user for each candidate sleeping time frame in a data set of glucose level values of the user obtained over time for a specified period for each of multiple days. Each of the candidate sleeping time frames may represent a duration in which the user may have been sleeping. Execution of the programming instructions further may cause the processor to determine a mean or median of the variability metric of the glucose level values in the data set for each candidate sleeping time frame across the days in the specified period, designate a selected one of the candidate sleeping time frames that has the lowest mean or median variability metric as the sleeping time frame for the user.

The programming instructions, when executed by the processor, may cause the processor to set the sleeping basal medicament delivery rate that is input to a control system of the medicament delivery system as an average of the basal medicament delivery rate for the sleeping time frame across the days in the specified period. The variability metric may be a standard deviation. Each candidate sleeping time frame may be between 5 hours to 12 hours in duration. The specified period may be a period of several consecutive days. The data set may contain data for 24 hours of each of the days in the specified period.

The programming instructions, when executed by the processor, may cause the processor to designate an awake basal medicament delivery rate that is input into a control system of the medicament delivery system for times in each day that are not part of the designated sleeping time frame. The awake basal medicament delivery rate that is input into a control system of the medicament delivery system may be an average basal medicament delivery rate for times that are not part of the designated sleeping time frame in the specified period. The storage, the processor, and the pump may be part of a medicament delivery device that is part of the medicament delivery system. The medicament delivery system may include a management device for managing the pump and wherein the processor and the storage are part of the management device.

In accordance with another inventive facet, a method performed by a processor of a medicament delivery system includes calculating with the processor a variability metric of glucose level values of a user of the medicament delivery system for each candidate sleeping time frame in a data set of glucose level values of the user obtained over time for a specified period for each day of multiple days. Each candidate sleeping time frame may represent a duration in which the user may have been sleeping. The method also may include determining with the processor a mean or median of the variability metric of the glucose level values for each of the candidate sleeping time frames across the days in the specified period and designating a selected one of the candidate sleeping time frames that has the lowest mean or median variability metric as the sleeping time frame.

The method may include setting the sleeping basal medicament delivery rate that is input into a control system of the medicament delivery system with the processor as an average of basal medicament delivery rate for the sleeping time frame across the days in the specified period. The variability metric may be a standard deviation. The method may include designating an awake basal medicament delivery rate that is input into a control system of the medicament delivery system for times in each day that are not part of the designated sleeping time frame. The processor may be part of a medicament delivery device that is part of the medicament delivery system or part of a management device for managing the medicament delivery device and is part of the medicament delivery system.

Programming instructions for performing the method may be stored in a non-transitory processor-readable storage medium.

### BRIEF DESCRIPTION OF THE DRAWINGS

FIG. 1 depicts a block diagram of a medicament delivery system of exemplary embodiments.

FIG. 2 depicts a flowchart of illustrative steps that may be performed in exemplary embodiments to determine and assign basal medicament delivery rates for a sleeping time frame and an awake time frame of a user.

FIG. 3 depicts a flowchart of illustrative steps that may be performed in exemplary embodiments to determine a most likely sleeping time frame.

FIG. 4 depicts a flowchart of illustrative steps that may be performed in exemplary embodiments to obtain a data set of glucose level data for a user to identify a sleeping time frame.

FIG. 5 depicts an illustrative data set used to identify a sleeping time frame in exemplary embodiments.

FIG. 6 depicts a flowchart of illustrative steps that may be performed in exemplary embodiments to determine a mean or median variability metric for a candidate time frame.

FIG. 7 depicts an illustration of calculation of median variability metrics for candidate sleeping time frames in exemplary embodiments.

FIG. 8 depicts an illustration of choosing a candidate sleeping time frame with a lowest median variability metric as the sleeping time frame for the user in exemplary embodiments.

FIG. 9 depicts a flowchart of illustrative steps that may be performed in exemplary embodiments to determine an hourly basal medicament delivery rate for a sleeping time frame.

FIG. 10 depicts a flowchart of illustrative steps that may be performed in exemplary embodiments to determine an hourly basal medicament delivery rate for an awake time frame.

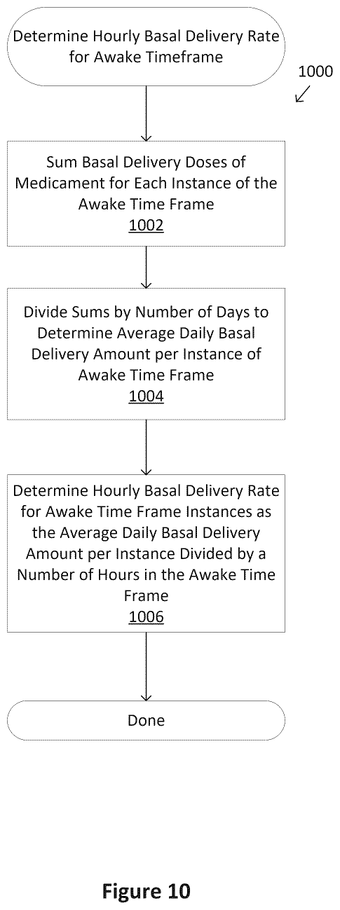

FIG. 11 depicts a flowchart of illustrative steps that may be performed in exemplary embodiments to change the basal medicament delivery rate during a sleeping time frame if a bolus of medicament is delivered.

FIG. 12A depicts an illustrative plot of standard deviation values of glucose levels of a first user over candidate sleeping time frames and total basal dose amounts delivered for the candidate sleeping time frames.

FIG. 12B depicts an illustrative plot of standard deviation values of glucose levels of a second user over candidate sleeping time frames and total basal dose amounts delivered for the candidate sleeping time frames.

### DETAILED DESCRIPTION

Exemplary embodiments may identify a sleeping time frame for a user of a medicament delivery device by analyzing glucose level data for the user in a historic data set, such as a week of glucose level data for the user. This enables a custom basal medicament delivery rate (aka “basal delivery rate”) to be assigned to the time frame that is known to be when the user typically sleeps. As a result, the basal medicament delivery rate to the user may be set lower during the sleeping time frame to reduce the risk of hypoglycemia and reduce negative glucose excursions relative to a target glucose level. The analysis may begin with defining candidate sleeping time frames. Candidate sleeping time frames are possible time windows in which the user may typically sleep. Each candidate sleeping time frame may be of a common fixed duration (such as seven or eight consecutive hours). For instance, a first candidate sleeping time frame may extend from 10 pm to 6 am, whereas a second candidate sleeping time frame may extend from 11 pm to 7 am. The glucose level data of the user in the historic data set for candidate sleeping time frames may be analyzed. The analysis may entail identifying a degree of variability of the glucose level data for each of the sleeping time frames by calculating a variability metric, such as a standard deviation, of glucose levels for the user in each candidate sleeping time frame. Values of the variability metric may be calculated for each candidate sleeping time frame for each of the multiple days in the data set. The average of the variability metric values over the multiple days may be calculated for each candidate sleeping time frame. The candidate sleeping time frame with the lowest variability as reflected in the variability metric average may be selected as the sleeping time frame for the user.

Once the sleeping time frame of the user has been identified, the basal delivery rate of medicament for the sleeping time frame may be determined. In some exemplary embodiments, basal medicament delivery dose data for the sleeping time frame over the multiple days may be gathered and summed. The basal medicament delivery dose sums for the sleeping time frame may then be averaged over the multiple days by summing all of the basal medicament delivery dose sums and dividing the resultant sum by the number of days in the multiple days. The average basal medicament delivery dose sum may then be divided by the number of hours in the sleeping time frame to yield an hourly basal medicament delivery rate for the sleeping time frame. This hourly delivery rate may be input to the control application and used as the basal medicament delivery rate by the control application of the medicament delivery device during the sleeping time frame. This rate subsequently may be adjusted during the sleeping time frame responsive to the glucose level of the user. Alternately, in some embodiments, there may be no adjustments during the sleeping time frame.

This approach yields an accurate estimate of when the user typically sleeps. Further, the basal medicament delivery rate reflects the historic basal medicament delivery data for the sleeping time frame. This represents a fasting basal medicament delivery rate for the user that is customized for the user.

After the sleeping time frame is determined, the awake time frame may be set as the time frame in a 24 hour period that is not the sleeping time frame. For instance, if the sleeping time frame is from 11 pm to 6:55 am, the awake time frame is from 7 am to 10:55 pm, if using a 5-minute interval cycle. The basal medicament delivery rate for the awake time frame may be set as the average hourly basal medicament delivery rate in the awake time frame over the multiple days. The awake time frame may have a higher basal medicament delivery rate than the sleeping time frame. This may reduce the risk of hyperglycemia and reduce the extent of positive excursions from the target glucose level during the awake time frame. As mentioned above these basal medicament delivery rates may be input to and subsequently adjusted by the control system during the awake time frame and sleeping time frame. In some exemplary embodiments, however, the rate may not be adjusted.

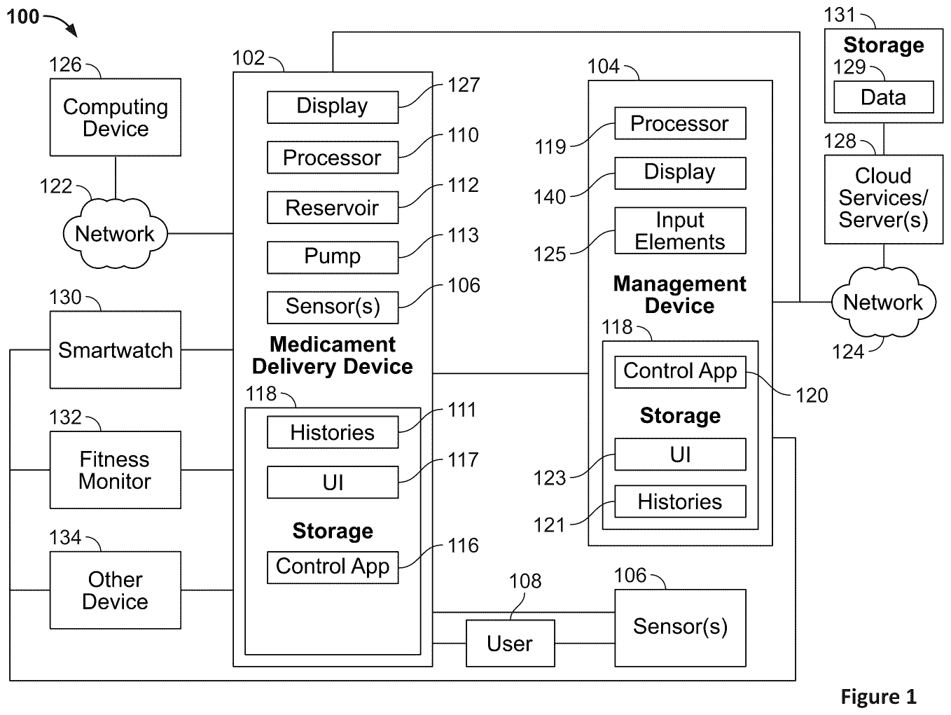

FIG. 1 depicts a block diagram of an illustrative medicament delivery system 100 that is suitable for delivering a medicament to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no tubes and an infusion location directly under the medicament delivery device 102, or carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application 116 may serve as a central part of the control system for the medicament delivery device 102. The control application 116 may use a glucose prediction model as described below for predicting future glucose levels of the user 108. The storage 114 may hold histories 111 for a user, such as a history of basal medicament deliveries, a history of bolus medicament deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. In some embodiments, a structure, such as part of the housing, may be provided for holding a vial or other source of medicament rather than including a reservoir 112. A fluid path to the user 108 may be provided, and the medicament delivery device 102 may expel the medicament from the reservoir 112 or other medicament source to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when the display is a touchscreen. The management device 104 may further include input elements 125, such as a keyboard, button, knobs, or the like, for receiving input of the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may deliver a first medicament, such as insulin, for lowering glucose levels of the user 108 and also deliver a second medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. More generally, the medicament delivery device 102 may deliver a medicament for managing and/or affecting glucose levels of the user 108. In other embodiments, the medicament delivery device 102 may deliver concentrated insulin. In some embodiments, the medicament or medicament delivered by the medicament delivery device may be a coformulation of two or more of those medicaments identified above. In an exemplary embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to be to offset the expected rise in glucose level of the user 108 from ingesting a meal or for correcting a persistently elevated glucose level (i.e., one that is persistently higher than a target glucose level). Boluses tend to be one time deliveries for offsetting a meal or for correcting a glucose level and tend to be larger than bolus insulin deliveries. Insulin boluses may be delivered manually by the user 108, such as via a syringe, or may, in some exemplary embodiments, be delivered by the medicament delivery device 102. Basal insulin doses tend to be smaller than insulin bolus doses and are delivered periodically, such as once each operational cycle of the control approach of the medicament delivery device 102 (e.g., every 5 minutes). The aim of the basal insulin deliveries is to keep the user's glucose level within a target range that is desirable using small ongoing insulin doses.

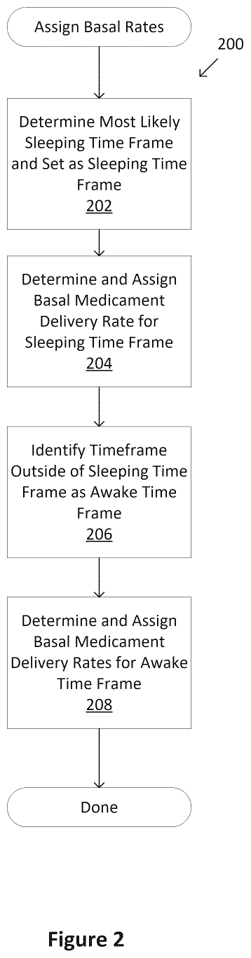

FIG. 2 depicts a flowchart 200 of illustrative steps that may be performed in exemplary embodiments to assign basal medicament delivery rates for a sleeping time frame when the user generally sleeps and an awake time frame when the user generally is awake. At 202, a most likely sleeping time frame is determined and that time frame is designated as the sleeping time frame. At 204, a basal medicament delivery rate is determined and then assigned to the sleeping time frame based on recent basal medicament delivery rates during the sleeping time frame. This basal medicament delivery rate may be adjusted over time during the sleeping time frame responsive to glucose levels of the user, as mentioned above. At 206, the time frame in a day that lies outside of the sleeping time frame is designated as the awake time frame. At 208, the basal medicament delivery rate is determined and then assigned to the awake time frame. The basal medicament delivery rate is determined from recent basal delivery rates for the awake time frame. In some embodiments, no new basal rate is assigned to the awake time frame, i.e., the process stops after step 204.

The steps of the methods of FIG. 2 may be performed periodically, such as once every few days, once a week, or once a month. The steps may, instead of being performed periodically, may be performed responsive to events, such before a new medicament delivery device 102 is activated to replace an old disposable medicament delivery device or when a new medicament delivery device 102 is activated or due to user input requesting an adjustment. Further, the steps may be performed to establish manual mode basal delivery rates for medicament, such as may be found in some open loop control modes.

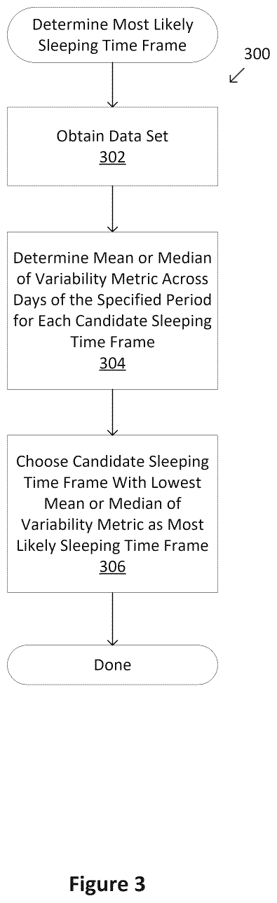

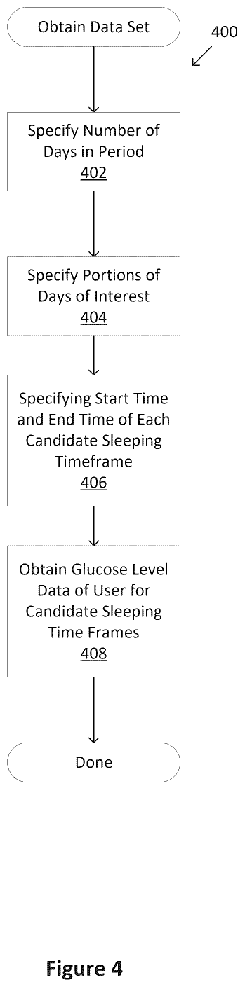

FIG. 3 depicts a flowchart 300 of illustrative steps that may be performed in exemplary embodiments to determine the most like sleeping time frame for a user 108. At 302, a data set of glucose level data for the user 108 is obtained. This data set may be part of the histories 111 or 121 stored on the medicament delivery device 102 or management device 104, respectively. The obtaining may entail accessing the data set in storage or receiving the data set. FIG. 4 depicts a flowchart 400 of illustrative steps that may be performed in exemplary embodiments to obtain the data set. As part of this obtaining, the metes and bounds of the times associated with the data set need to be specified. At 402, the number of days in the specified period of the data set is specified. This may be a tunable parameter or may be specified by the control application 116 or 120. Alternatively, the number of days in the specified period may be a default value or an initial value that may be modified. Examples of the number of days in the specified period include 1 day, 2 days, 7 days, etc. In some embodiments, the number of days is between 1 day to 14 days, more specifically between 2 days to 10 days and in particular between 3 days to 7 days. At 404, the portions of the day that are of interest (i.e., for which data is to be obtained) may be specified. The portions of day that are of interest may be a tunable parameter or may be specified by the control application 116 or 120. In some embodiments, the portions of day that are of interest include nighttime and optionally the adjoining morning and/or evening periods. The portions of the day that are of interest may also be entered by user input, for example, if the user works night shifts. This specification may indicate that all 24 hours of each day are of interest or that only a portion of the day is of interest, such as between 9 pm and 8 am. At 406, the start times and end times of each candidate sleeping time frame may be specified. Generally, the candidate sleeping time frames may be of a length that corresponds to an average time of sleep for a user (such as between 6 hours to 9 hours or more broadly between 5 hours and 12 hours). In some embodiments, the a length that corresponds to an average time of sleep for a user is between 4 hours to 13 hours, more specifically between 5 hours to 12 hours and in particular between 6 hours to 9 hours. At 408, the glucose level data of the user 108 is obtained for the candidate sleeping time frames.

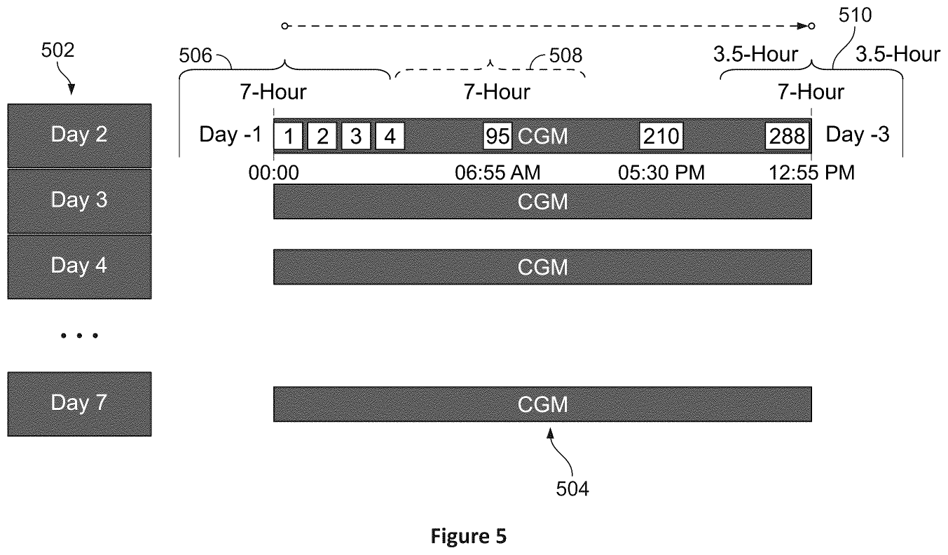

With reference again to the flowchart of FIG. 3, at 304, the mean of the variability metric values across the days of the specified period of the data set may be determined for each candidate sleeping time frame. In some embodiments, the median rather than the mean may be determined, especially if outliers are anticipated that may skew the mean. The variability metric may be, for example, a standard deviation or other measure of variability, such as variance, or interquartile range. FIG. 5 depicts an illustrative data set. The data set contains glucose level data for six days 502, labelled as day 2 through day 7. The data set includes glucose level data 504 for each of these days 502. In this example, each candidate sleeping time frame 506, 508 and 510 is 7 hours in length. The first candidate sleeping time frame 506 is centered at midnight and extends 3.5 hours before midnight and 3.5 hours after midnight. The second candidate sleeping time frame 508 may start where the first candidate sleeping time frame 506 stops. This temporal spacing of the sleeping candidate time frames is suitable for a medicament delivery device that has 5 minute operational cycles and includes a glucose level reading for each cycle. Hence, there are 12 glucose level readings per hour and 288 glucose level readings per day (not depicted to scale). The cycle lengths may be adjusted, for example, between about 3 minutes to about 10 minutes or between other ranges of time, such as 15 minutes, etc.

In some exemplary embodiments, the candidate sleeping time frames may overlap. For instance, a candidate sleeping time frame may start every five minutes or another period representing an operational cycle of the medicament delivery device. As a result there are 288 candidate sleeping time frames in that instance. This approach has the benefit of being exhaustive so that every possible candidate sleeping time frame in each day is considered.

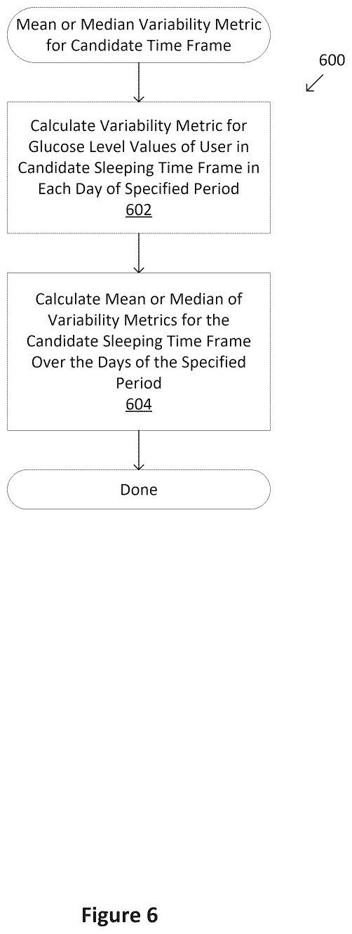

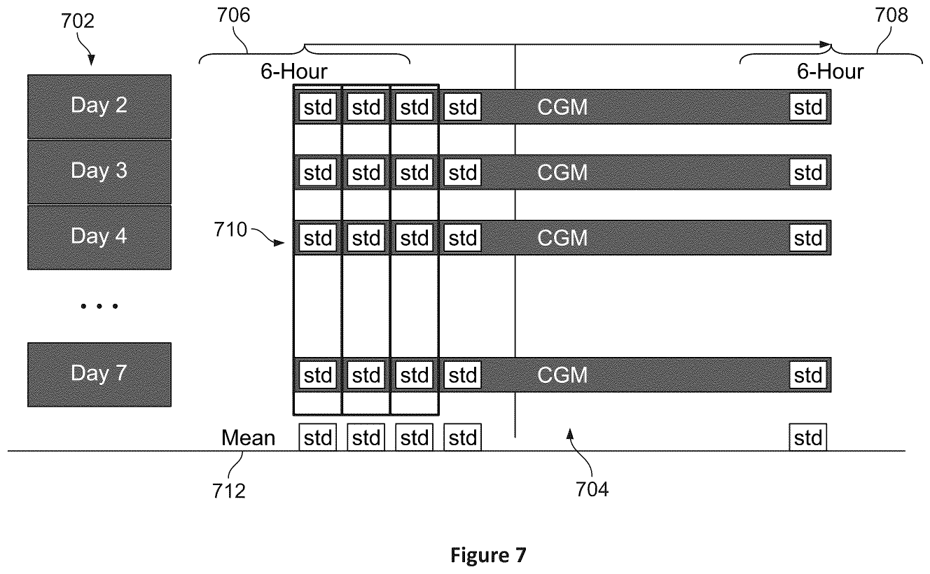

FIG. 6 depicts a flowchart 600 of illustrative steps that may be performed in exemplary embodiments to determine the mean or median of the variability metric values. These steps will be described herein in conjunction with an illustrative data set as shown in FIG. 7. At 602, the variability metric is calculated for glucose level values of the user in the candidate sleeping time frames in each day of the specified period. The aim is to find the candidate sleeping time frame that has the lowest variability as that time frame most likely corresponds to when the user 108 sleeps. As shown in FIG. 7, for the days 702, glucose level data 704 is processed for each of 288 candidate sleeping time frames (e.g., 706, and 708) to calculate the variability metrics 710 for those candidate sleeping time frames (see “std”, which designates standard deviation values in FIG. 7). At 604, the mean or median of the variability metrics of the candidate sleeping time frames over the days of the specified period are calculated. Thus, in FIG. 7, the means 712 of the standard deviations over the 6 days for each candidate sleeping time frame are calculated.

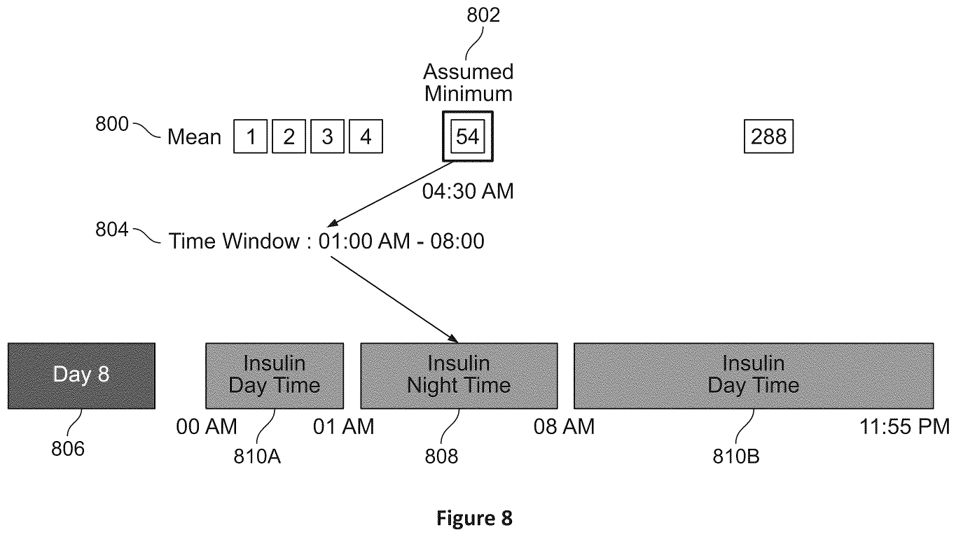

Then, at 306 (see FIG. 3), the candidate sleeping time frame with the lowest mean or median variability metric value is chosen as the most likely sleeping time frame. When a user sleeps, the user does not eat and is not active. As a result, the variability in their glucose level is low relative to periods when the user is awake. FIG. 8 depicts an example. The means 800 of the variability metrics for the candidate sleeping time frames across the days of the specified period are calculated. The minimum in the example shown in FIG. 8 is candidate sleeping time frame 802, which is the 54^{th} of the 288 candidate sleeping time frames. The candidate sleeping time frame 802 corresponds to a time window 804 between 1:00 am and 8:00 am. The awake time frame includes from midnight to 1:00 am (see 810A) and from 8:00 am to midnight (see 810B).

As mentioned above, at 202, the most likely sleeping time frame is designated as the sleeping time frame. Hence, as shown in FIG. 8, for the next day (i.e., day 8), the sleeping time frame 808 is between 1:00 am and 8:00 am.

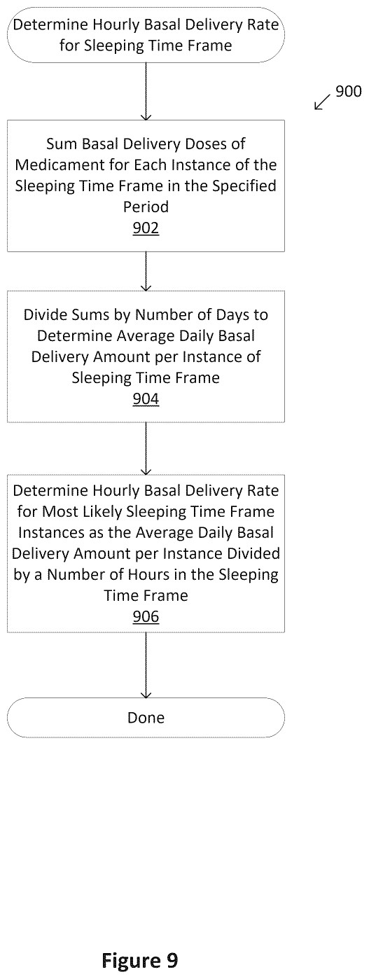

Once the sleeping time frame is designated, the basal medicament delivery rate for the sleeping time frame may be determined (see 204). The basal delivery rate may be, for instance, an hourly basal medicament delivery rate. FIG. 9 depicts a flowchart 900 of illustrative steps that may be performed in exemplary embodiments to determine the basal delivery rate for the sleeping time frame. At 902, the basal delivery doses of medicament that were delivered to the user 108 during the sleeping time frame over the days of the specified period may be summed. At 904, the sums may be divided by the number of days in the specified period to determine an average daily delivery amount of the medicament during the sleeping time frame. At 906, the average daily delivery amount of medicament to the user during the sleeping time frame for the days in the specified period may be divided by the number hours in the sleeping time frame to determine the hourly basal delivery rate of the medicament to the user 108 for the sleeping time frame.

It should be appreciated that the historic basal delivery dose data that is used need not be determined over the same time window as that of the glucose level data set that was used to determine the sleeping time frame. In addition, instead of summing doses for the sleeping time frame for each day, the control application 116 or 120 may calculate average hourly delivery rates for the candidate time frame for each day and take the average daily delivery rate for the sleeping time frame over the days as the delivery rate for the sleeping time frame.

An hourly basal medicament delivery rate may be determined for the awake time frame. The approach for determining this rate largely may be like that used for determining the rate for the sleeping time frame. At 1002, the basal delivery doses of medicament are summed for each instance of the awake time frame in the specified period. At 1004, the sums may then be divided by the number of days in the specified period to determine average daily basal delivery amounts per instance of the awake time frame. At 1006, the hourly basal medicament basal delivery rate may be determined by dividing the average daily basal medicament delivery amount by a number of hours in the awake time frame.

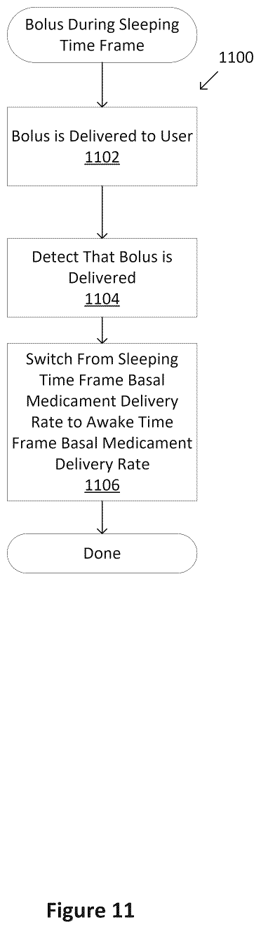

One risk with setting a separate basal medicament delivery rate for a sleeping time frame is that the user 108 may eat during a time period during which they usually sleep. The risk of hyperglycemia may increase given the lower sleeping basal medicament delivery rate. Hence, the exemplary embodiments may perform the steps of the flowchart 1100 of FIG. 11. The process may begin with a bolus of medicament being delivered at 1102. The bolus may be manually delivered or automatically delivered by the medicament delivery device 102. The system may detect that the bolus has been delivered at 1104. The detection may be responsive to user input or by a notification from the control application 116 or 120 that a bolus has been delivered. The delivery of the bolus may act as a signal that a meal or snack has been consumed. The calculation of the bolus dose by the user or the control application 116 or 120 may have assumed that the basal medicament delivery rate is that of the awake time frame. Thus, at 1106, the control application 116 or 120 may switch from the sleeping time frame basal medicament delivery rate to the awake time frame medicament delivery rate to reduce the risk of hyperglycemia. This basal medicament delivery rate may be used for the next few hours (e.g., 3 or 4 hours). Alternatively, this basal medicament delivery rate may be used until the following sleeping time frame.

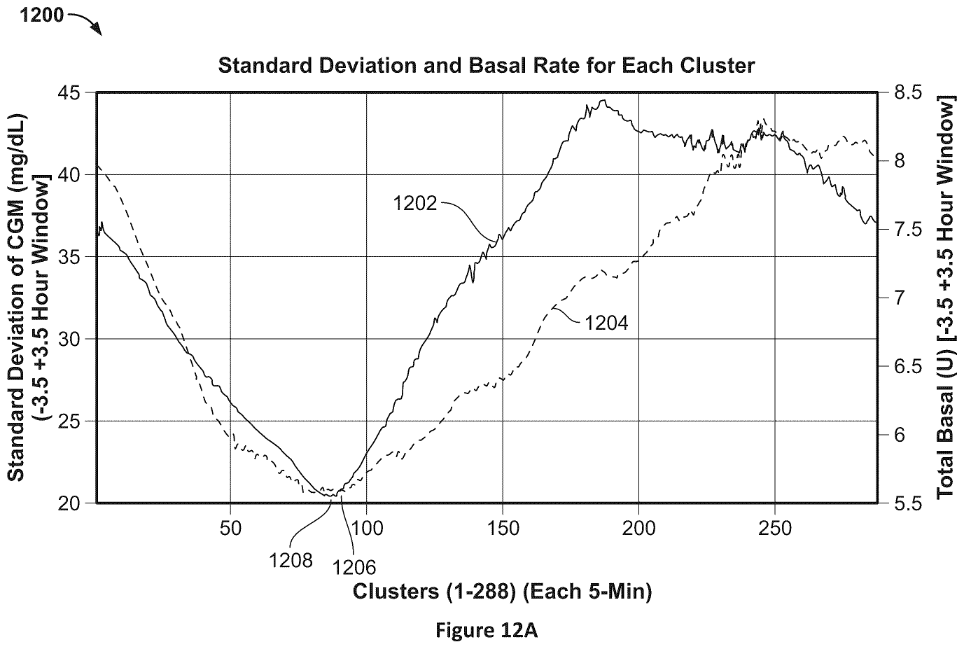

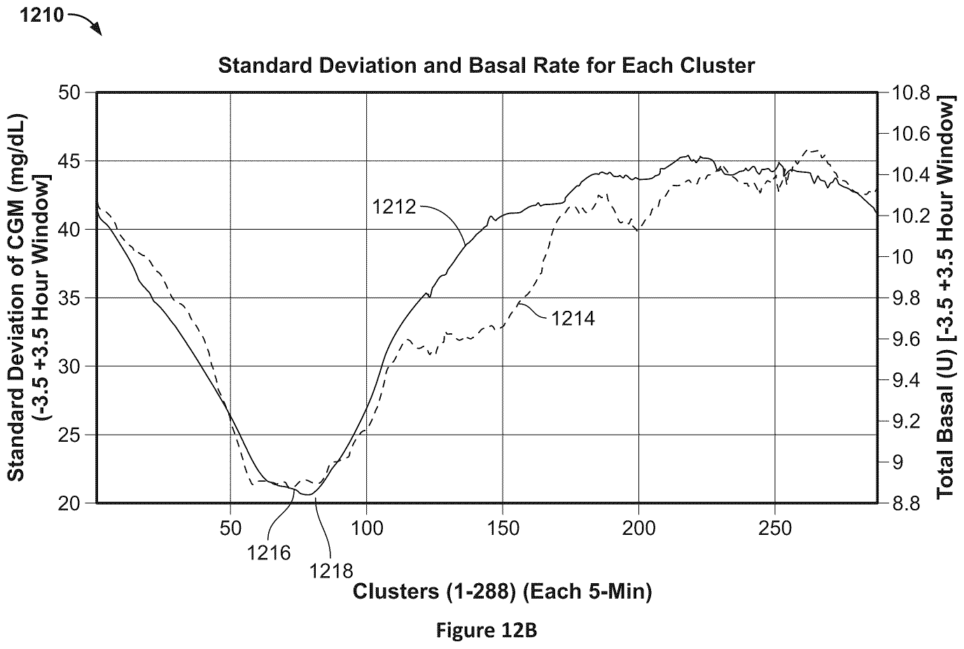

FIG. 12A depicts a plot 1200 of the standard deviation value 1202 for each candidate time frame (a “cluster”) and the basal medicament delivery dose amounts 1204 delivered to a first user. FIG. 12B depicts a plot 1210 of the standard deviation value 1212 for each candidate time frame and the basal medicament delivery dose amounts 1214 for a second user. These plots 1200 and 1210 evidence that the standard deviation of the glucose level values are strongly correlated with total basal insulin usage and trend together. Further, the minimum point of the standard deviation (see 1206 and 1216) is closely located relative to the minimum point of total basal insulin usage (see 1208 and 1218), which is the fasting/nighttime basal rate of the users.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage. The methods disclosed herein may be computer-implemented methods.

While exemplary embodiments have been described herein, various changes in form and detail may be made without departing from the intended scope as defined in the appended claims and equivalents thereof.

1. A medicament delivery system, comprising:
a pump for delivering a medicament to a user;
a storage for storing programming instructions; and
a processor configured for executing the programming instructions to cause the processor to:
calculate a variability metric of glucose level values of the user for each candidate sleeping time frame in a data set of glucose level values of the user obtained over time for a specified period for each day of multiple days, wherein each of the candidate sleeping time frames represents a duration in which the user may have been sleeping,
determine a mean or median of the variability metric of the glucose level values in the data set for each candidate sleeping time frame across the days in the specified period, and
designate a selected one of the candidate sleeping time frames that has the lowest mean or median variability metric as a sleeping time frame for the user.

2. The medicament delivery system of claim 1, wherein the programming instructions when executed by the processor further cause the processor to set the sleeping basal medicament delivery rate that is input to a control system of the medicament delivery system as an average of basal medicament delivery rate for the sleeping time frame across the days in the specified period.

3. The medicament delivery system of claim 1, wherein the variability metric is a standard deviation.

4. The medicament delivery system of claim 1, wherein each candidate sleeping time frame is between 5 hours to 12 hours in duration.

5. The medicament delivery device of claim 1, wherein the specified period is a period of several consecutive days.

6. The medicament delivery system of claim 1, wherein the data set contains data for 24 hours of each of the days in the specified period.

7. The medicament delivery system of claim 1, wherein the programming instructions when executed by the processor further cause the processor to designate an awake basal medicament delivery rate that is input to a control system of the medicament delivery system for times in each day that are not part of the designated sleeping time frame.

8. The medicament delivery system of claim 7, wherein the awake basal medicament delivery rate that is input into a control system of the medicament delivery system is an average basal rate for times that are not part of the designated sleeping time frame in the specified period.

9. The medicament delivery system of claim 1, wherein the storage, the processor and the pump are part of a medicament delivery device that is part of the medicament delivery system.

10. The medicament delivery system of claim 1, further comprising a management device for managing the pump and wherein the processor and the storage are part of the management device.

11. A method performed by a processor of a medicament delivery system, comprising:
with the processor, calculating a variability metric of glucose level values of a user of the medicament delivery system for each candidate sleeping time frame in a data set of glucose level values of the user obtained over time for a specified period for each day of multiple days, wherein each candidate sleeping time frame represents a duration in which the user may have been sleeping;
with the processor, determining a mean or median of the variability metric of the glucose level values for each of the candidate sleeping time frames across the days in the specified period;
with the processor, designating a selected one of the candidate sleeping time frames that has the lowest mean or median variability metric as a sleeping time frame for the user.

12. A method of claim 11, further comprising, with the processor, setting the sleeping basal medicament delivery rate that is input into a control system of the medicament delivery system as an average of basal rate for the sleeping time frame across the days in the specified period.

13. The method of claim 11, wherein the variability metric is a standard deviation.

14. The method of claim 11, further comprising designating an awake basal medicament delivery rate that is input into a control system of the medicament delivery system for times in each day that are not part of the designated sleeping time frame.

15. The method of claim 11, wherein the processor is part of a medicament delivery device that is part of the medicament delivery system or part of a management device for managing the medicament delivery device and is part of the medicament delivery system.

16. A non-transitory processor-readable storage medium storing programming instructions that when executed by a processor cause the processor to:
calculate a variability metric of glucose level values of a user of a medicament delivery system for each candidate sleeping time frame in a data set of glucose level values of the user obtained over time for a specified period for each day of multiple days, wherein each of the candidate sleeping time frames represents a duration in which the user may have been sleeping;
determine a mean or median of the variability metric of the glucose level values of the user for each candidate sleeping time frame across the days in the specified period;
designate a selected one of the candidate sleeping time frames that has the lowest mean or median variability metric as a sleeping time frame for the user.

17. The non-transitory processor-readable storage medium of claim 16, wherein the programming instructions when executed by the processor further cause the processor to set the sleeping basal medicament delivery rate that is input to a control system of the medicament delivery system as an average of basal medicament delivery rate for the sleeping time frame across the days in the specified period.

18. The non-transitory processor-readable storage medium of claim 16, wherein the variability metric is a standard deviation.

19. The non-transitory processor-readable storage medium of claim 16, wherein the programming instructions when executed by the processor further cause the processor to designate an awake basal medicament delivery rate that is input into a control system of the medicament delivery system for times in each day that are not part of the designated sleeping time frame.

20. The non-transitory processor-readable storage medium of claim 16, wherein the processor is part of a medicament delivery device that is part of the medicament delivery system or part of a management device for managing the medicament delivery device and is part of the medicament delivery system.
